(19) United States
(12) Patent Application Publication
(10) Pub. No.: US 2016/0206745 A1
(43) Pub. Date: Jul. 21, 2016

(54) THREE DIMENSIONAL SOY PROTEIN-CONTAINING SCAFFOLDS AND METHODS FOR THEIR USE AND PRODUCTION

(71) Applicant: Northwestern University, Evanston, IL (US)

(72) Inventors: Karen B. Chien, Chicago, IL (US); Ramille N. Shah, Chicago, IL (US)

(21) Appl. No.: 14/980,132

(22) Filed: Dec. 28, 2015

Related U.S. Application Data
(62) Division of application No. 14/057,479, filed on Oct. 18, 2013, now abandoned.
(60) Provisional application No. 61/716,122, filed on Oct. 19, 2012.
(60) Provisional application No. 61/767,616, filed on Feb. 21, 2013.

(51) Int. Cl. A61K 47/42; C12N 5/00

(52) U.S. Cl. CPC A61K 47/42; C12N 5/0068; C12N 2533/50; C12N 2535/00; C12N 2537/10; B33Y 10/00

(57) ABSTRACT

Porous soy protein-based scaffolds and methods for making the scaffolds using 3D printing techniques are provided. Also provided are tissue growth scaffolds comprising the porous soy protein-based scaffolds and methods for growing tissue on the tissue growth scaffolds. The porous soy protein-containing scaffold comprises a plurality of layers configured in a vertical stack, each layer comprising a plurality of strands comprising denatured soy proteins.

### CROSS-REFERENCE TO RELATED APPLICATIONS

This application is a divisional of U.S. patent application Ser. No. 14/057,479 filed on Oct. 18, 2013, the entire contents of which are hereby incorporated by reference; which claims priority from U.S. provisional patent application Ser. No. 61/716,122 filed on Oct. 19, 2012, and from U.S. provisional patent application Ser. No. 61/767,616 that was filed on Feb. 21, 2013, both of which are incorporated herein by reference.

### BACKGROUND

Rapid prototyping, such as solid free-form fabrication, is a technique used to form porous three-dimensional (3D) scaffolds for tissue engineering applications. A major advantage of this fabrication method is the ability to control pore structures and geometries. 3D-Bioplotting is a method in which a viscous or a paste-like slurry is extruded by compressed air-induced pressure, onto a surface in air or submerged in a liquid medium (1-4). The continuous injection of a material using layer-by-layer deposition generates a fully interconnected pore structure. Although solid free form fabrication is commonly applied to synthetic materials, 3D printing and plotting of natural biopolymers such as proteins and polysaccharides is not as common. One challenge in printing soft materials is that they have a wide range of intrinsic properties such as viscosity, which can vary greatly between batches during sample fabrication.

Animal and plant-based proteins are ideal biomaterials due to inherent bioactivity, degradation properties, and natural binding sites that can be tailored to control cell adhesion and growth both in vitro and in vivo (5). Specific optimization and design of printing parameters and conditions have been developed for various proteins (6-10) and composites (11, 12). 3D printing using a powder mixed with a binding solution was used to fabricate a blend of corn starch, gelatin, and dextran (11). Gelatin alone has also been plotted at 3% and 10% concentrations in water (6, 7). Landers et al. discussed the important fabrication parameters involved in plotting soft gel materials including viscosity, swelling in plotting medium, density, and thermal behavior of the plotted material, using gelatin and agar as examples (7). Bovine collagen has been fabricated using an indirect printing technique, where the slurry is printed into a negative mold, and the mold is subsequently dissolved away after freeze-drying to leave a scaffold with a predefined structure (8-10). The 3D Bioplotter was also used to fabricate 3D collagen-chitosan-hydroxyapatite hydrogels in a study characterizing the angiogenic and inflammatory response in vivo in comparison with a plotted PLGA scaffold (12).

Two main challenges with printing natural biopolymers include controlling strut solidification upon extrusion (6) and electrostatic interactions between the biopolymer and liquid media if plotting into a solution. Scaffolds formed in air would require that the slurry dries at an appropriate rate during extrusion to support subsequent printed layers. Plotting into a liquid medium requires that the density of the solution be the same as the material being injected to preserve strand shape and to prevent dissolution of the printed strands (2, 7). Standardizing the plotting method for individual soft materials is essential since variability in moisture content of slurries and drying as a result of variable environmental conditions can affect reproducibility during mass production.

### SUMMARY

Three dimensional porous soy protein-containing scaffolds comprising denatured soy proteins are provided. The soy protein chains in the denatured soy proteins can be crosslinked with enzymatic crosslinkers or with non-enzymatic chemical crosslinkers. The scaffolds can be tailored to have pore sizes suitable for promoting cell growth and proliferation within the pores and/or robust mechanical properties, as determined by their compressive moduli.

In one aspect, a porous soy protein-containing scaffold comprises a plurality of layers configured in a vertical stack, each layer comprising a plurality of strands comprising denatured soy proteins.

In some embodiments, the scaffold has a porosity of at least 50% and a pore interconnectivity of at least 90%.

In some embodiments, the scaffold is configured such that within each layer the plurality of strands are spaced apart and aligned along their longitudinal axes and the angle, θ, defined by the longitudinal axes of the strands in adjacent layers is in the range of 0°<0≦90°, such that pores are defined by the strands in adjacent layers of the vertical stack. In some embodiments, the angle θ is in the range of 45°≦θ≦90° or in the range of 75°≦θ≦90°. In some embodiments, the angle θ in the scaffold is in the range of 85°≦θ≦90°; the soy protein chains in the denatured soy proteins are crosslinked; the crosslinking density of the soy protein chains within the scaffolds is at least 0.3; and the scaffold has a compressive modulus of at least 3500 Pa.

In some embodiments, the scaffold has a compressive modulus of at least 1000 Pa or a compressive modulus in the range from about 2000 Pa to about 5000 Pa.

In some embodiments, the soy protein chains in the denatured soy proteins are crosslinked and the relative crosslinking density of the soy protein chains within the scaffolds is at least 0.1 or in the range from about 0.1 to about 0.35.

In some embodiments, the pores have a median pore diameter in the range from about 200 μm to about 1000 μm or in the range from about 300 μm to about 400 μm.

In some embodiments, the median x-axis strand thickness for the strands is no greater than about 600 μm.

In some embodiments in which the scaffold is crosslinked, the crosslinks comprise carbodiimide crosslinks.

In some embodiments, the scaffold further comprises at least one of a growth factor or a drug incorporated into one or more of the strands.

In another aspect, a tissue growth scaffold comprises any of the disclosed porous soy protein-containing scaffolds and tissue-forming cells, or cells that are precursors to tissue-forming cells, integrated within the pores of the porous soy protein-containing scaffold.

In another aspect, a method of growing tissue on a tissue growth scaffold comprises culturing the scaffold in a cell growth culture medium, wherein the scaffold comprises a plurality of layers configured in a vertical stack, each layer comprising a plurality of strands comprising denatured soy proteins; and tissue-forming cells, or cells that are precursors to tissue-forming cells, integrated within the pores of the scaffold.

In another aspect, a method of forming a porous soy-protein containing scaffold comprises extruding a slurry comprising denatured soy proteins in the form of a first layer, the first layer comprising a plurality of strands; and extruding the slurry in the form of one or more additional layers, each additionally layer being vertically stacked upon the previously extruded layer and comprising a plurality of strands.

In some embodiments, the strands in each layer are spaced apart and aligned along their longitudinal axes, and the angle, θ, defined by the longitudinal axes of the strands in adjacent layers is in the range of 0°<θ<90°.

In some embodiments, the slurry further comprises water and a plasticizer.

In some embodiments, the method further comprises dehydrating the scaffold in an alcohol.

In some embodiments, the method further comprises removing water from the scaffold via a dehydrothermal treatment. In some embodiments, the method further comprises freeze-drying the scaffold, whereby water is removed via the sublimation of water frozen on the strand surfaces, prior to the dehydrothermal treatment.

In some embodiments, the method further comprises chemically or enzymatically crosslinking the soy protein chains in the denatured soy proteins.

In some embodiments, at least 9 additional layers are extruded.

In some embodiments, the slurry comprises at least one of a growth factor or a drug.

In another aspect, a method of forming a porous biopolymer-containing scaffold comprises extruding a slurry comprising a biopolymer in the form of a first layer, the first layer comprising a plurality of strands; and extruding the slurry in the form of one or more additional layers, each additionally layer being vertically stacked upon the previously extruded layer and comprising a plurality of strands; wherein the mass flow rate of the slurry is maintained at a constant rate during extrusion by adjusting one or both of the extrusion pressure and extrusion speed during extrusion.

In some embodiments, the strands in each layer are spaced apart and aligned along their longitudinal axes, and the angle, θ, defined by the longitudinal axes of the strands in adjacent layers is in the range of 0°<θ<90°.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings.

FIGS. 1A-1B show idealized schematics of Bioplotted scaffolds. FIG. 1A Definitions of the angular placement of layers (θ), pore width (or strand spacing) (w), strand thickness (t), layer spacing (s), and strand thickness in the z-direction (z). s can be less than, equal to, or greater than z (s≧z for natural biopolymers), and z=t if the strand has a perfectly circular cross-section. FIG. 1B CAD images of top and cross-section views of idealized 45° (left) and 90° (right) scaffolds.

FIG. 2 shows the mass flow rate of soy protein slurries of varying soy protein and glycerol concentration measured using the BioPlotter. Mass was extruded at a pressure of 3 bars for 2 seconds (N=3 measurements per temperature). All error bars represent one standard deviation.

FIG. 3A. Mass flow rate of 20% soy protein, 4% glycerol slurry with and without the addition of 7.5 mM DTT. Schematics of the protein strand packing during slurry ejection through the needle in the −DTT and +DTT cases are provided to the left of each of the data series. FIGS. 3B, 3C. Macroscopic images of slurries printed at 27° C. with the addition of 2% trypan blue (circle diameter set at 10 mm). Printing pressure was adjusted so that both circles were printed with a flow rate of 0.02 g/s. FIG. 3B. Slurry without DTT. FIG. 3C. Slurry with the addition of DTT. FIGS. 3D, 3E. SEM images of the surfaces of the printed strands from the macroscopic images. FIG. 3D. Slurry without DTT. FIG. 3E. Slurry with the addition of DTT.

FIG. 4E. Mass flow rate of a 20% soy protein, 4% glycerol slurry at various pressures (n=3 measurements per temperature). Letters A-D represent mass flow rates with corresponding macroscopic images of strand shape. FIG. 4A. Pressure less than optimal plotting pressure. FIG. 4B. Optimal plotting pressure to produce well-defined pore structures and z-pores. FIGS. 4C, 4D. Pressures over the optimal plotting pressure.

FIGS. 5A-5F show the effect of post treatment on scaffold surface morphology. FIG. 5A. Representative SEM image of scaffold surface showing pore structure of 90° scaffold. FIG. 5F. Mmacroscopic views of 45° scaffolds with various post-treatments. N=5 for all diameters measured. NT: no further treatment beyond 95% ethanol dehydration. Average diameter was 6.57±0.19 mm. FD-DHT: scaffolds freeze-dried before dehydrothermal treatment. Average diameter was 6.57±0.14 mm. DHT: dehydrothermal treatment. Average diameter was 4.97±0.33 mm. EDC: carbodiimide crosslinking. Average diameter was 6.09±0.04 mm. FIGS. 5B-5D. SEM images of the strand surface after various post-treatments: (FIG. 5B) NT, (FIG. 5C) EDC, (FIG. 5D) DHT, (FIG. 5E) FD-DHT.

FIGS. 6A-6D show the effect of post-treatment on structural, mechanical, and degradation properties of BioPlotted soy scaffolds. All error bars represent one standard deviation. FIG. 6A. Crosslink density of scaffolds (N=5-6). FIG. 6B. Mass loss of scaffolds upon rinsing 3× in PBS (N=4). FIGS. 6C, 6D. Compressive moduli of scaffolds plotted with various angles (N=5). *: P<0.05; **: P<0.01; ***: P<0.001.

FIGS. 7A-7B show the effect of post-treated scaffolds on human mesenchymal stem cell seeding efficiency. FIG. 7A. Cell seeding efficiency (%) of the scaffolds with starting seeding density of 1×10⁶ cells/scaffold. FIG. 7B. Proliferation of cells on scaffolds at days 1 and 7. *: P<0.05.

### DETAILED DESCRIPTION

Porous soy protein-based scaffolds and methods for making the scaffolds using 3D printing techniques are provided. Also provided are tissue growth scaffolds comprising the porous soy protein-based scaffolds and methods for growing tissue on the tissue growth scaffolds.

The porous soy protein-containing scaffolds can be used as tissue growth scaffolds by integrating tissue-forming cells, or cells that are precursors to tissue-forming cells, within the pores or within the struts of the porous soy protein-containing scaffolds. Tissue can be grown within the tissue growth scaffolds by incorporating tissue-forming cells, or cells that are precursors to tissue-forming cells, into the porous soy protein-containing scaffolds (as by, for example, seeding); and culturing the seeded scaffolds in a cell growth culture medium. Human mesenchymal stem cells, hematopoetic stem cells, embryonic stem cells, and induced pluripotent stem cells are examples of precursors to tissue-forming cells. Examples of tissue-forming cells include osteoblasts, chondrocytes, fibroblasts, endothelial cells, and myocytes.

The porous soy protein-containing scaffolds can be printed via layer-by-layer extrusion of a soy protein-containing slurry (or “ink”) through a print head in a printer, such as an inkjet-type printer or bioplotter. In addition to the denatured soy proteins, the slurry may comprise a carrier liquid, such as water and additives, including plasticizers (e.g., glycerol) and antibiotic or antimycotic agents. In some embodiments, the slurry comprises a growth factor or a drug (i.e., a pharmaceutical compound), such that the growth factor or drug can be incorporated directly into one or more of the strands during the printing of the scaffold. Various amounts of denatured soy protein may be used in the slurries. In some embodiments, the amount of denatured soy protein in the slurry is equivalent to the saturation point of the denatured soy protein. In some embodiments, the amount of denatured soy protein in the slurry is in the range from about 15 weight % to about 20 weight %.

The use of 3D printing for the fabrication of the scaffolds is advantageous because it provides for regular geometric patterning of the layers that make up the scaffold, which makes it possible to control and tailor the porosity, pore size and pore interconnectivity of the scaffold. The printing can be carried out at relatively low extrusion temperatures, including temperatures in the range from about room temperature (i.e., 21° C.) to about 40° C. The mass flow rate of the slurry during extrusion is a parameter for achieving optimal, reproducible scaffolds. The optimal mass flow rate generally depends upon the composition of the slurry, e.g., the concentration of the soy protein. For a particular composition, an optimal mass flow rate can be determined by first optimizing the extrusion speed (i.e., the time over which the extrusion occurs) and next, optimizing the extrusion pressure (i.e., the pressure applied to extrude the slurry) as discussed in the Example, below. The flow rate measured at the optimized extrusion speed and optimized extrusion pressure corresponds to an optimal mass flow rate which can be maintained at constant rate during extrusion by adjusting one or both of the extrusion pressure and extrusion speed during extrusion. This constant mass flow rate approach to 3D printing can also be applied to the printing of porous scaffolds comprising other biopolymers, such as other proteins or polysaccharides.

One embodiment of a method of fabricating a soy protein-containing scaffold via a 3D printing process comprises the following steps: extruding a slurry comprising denatured soy proteins in the form of a first layer, the first layer comprising a plurality of strands comprising the denatured soy proteins; extruding the slurry comprising denatured soy proteins in the form of a second layer over the first layer, the second layer comprising a plurality of strands comprising the denatured soy proteins. This layer-by-layer printing technique can be repeated by the stepwise extrusion of one or more additional layers until a scaffold having the desired thickness comprising the desired number of layers has been fabricated. For example, in some embodiments the vertical stacks comprise at least 10 layers, at least 20 layers or at least 100 layers. The strands within each layer may be printed in a regular, repeating pattern, may be printed in a random arrangement, or in some combination of both.

The term “strand” as used herein refers to an elongated, continuous and unbranched structure that is delineated and distinguishable as a distinct unit within the scaffold (e.g., as seen in a scanning electron microscope (SEM) image), although strands in neighboring layers in the stacked structure may be merged at their interfaces. Within the scaffolds, strands deposited in a given layer remain substantially within that layer, although there may be sagging of the strands in a layer into the underlying layer. Thus, the strands do not form a mesh that is entangled in three dimensions with strands extended through multiple stacked layers of the scaffold.

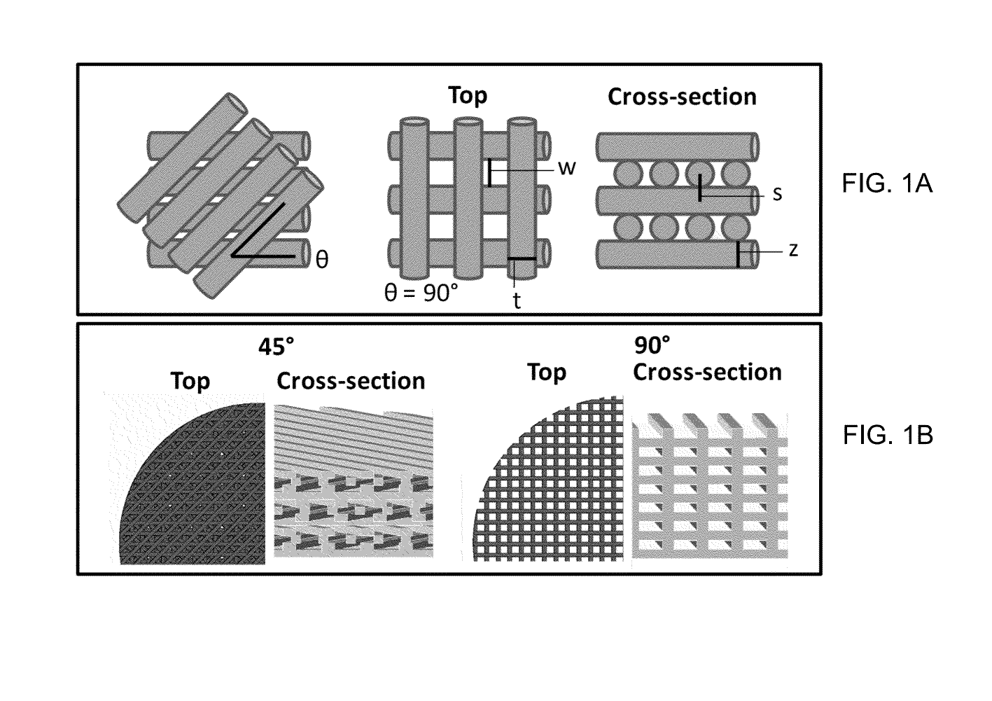

The strands in a given layer are spaced apart, defining a strand spacing between adjacent strands. (See “w” in FIG. 1A.) Typical strand spacings include those in the range from about 50 μm to about 1000 μm, which includes strand spacings in the range from about 1000 μm to about 500 μm or from about 200 μm to about 300 μm. The strand spacing may refer to an average or median strand spacing, by which it is meant the average/median of the spacing between adjacent strands within a given layer or within the entire scaffold. Strands in a given layer maybe aligned such that the strand spacing between adjacent strands is substantially the same along the lengths of the adjacent strands. Strands in a given layer may be aligned such that the longitudinal axes of adjacent strands are substantially parallel. However, the longitudinal axes of adjacent strands may be aligned without being substantially parallel, e.g., when the longitudinal axes of adjacent strands adopt substantially the same curved shape. Strands in a given layer are typically not physically connected to other another, although strands in neighboring layers may be merged at their interfaces.

The strands may be characterized by a thickness. The thickness may refer to the largest dimension (e.g., diameter) across a lateral cross-section of a strand. Alternatively, the thickness may refer to an x-axis thickness or a z-axis thickness. A x-axis thickness may refer to the largest dimension across a lateral cross-section of a strand as measured along an axis running parallel to a substrate over which the strand is printed. (See “t” in FIG. 1A.) A z-axis thickness may refer to the largest dimension across a lateral cross-section of a strand as measured along an axis running perpendicular to a substrate over which the strand is printed. (See “z” in FIG. 1A.) Typical thicknesses include those in the range from about 100 μm to about 1000 μm, which includes thicknesses in the range from about 100 μm to about 600 μm or from about 100 μm to about 300 μm. The thickness/x-axis thickness/z-axis thickness/diameter may refer to an average or median value, by which it is meant the average/median of the relevant parameter for the strands within a given layer or within the entire scaffold. The printing methods make it possible to form strands having uniform or substantially uniform thickness/diameters along their length. For example, in some embodiments the thickness/diameter of the strands deviates by no more than ±30% along their length. This includes embodiments in which the thickness/diameter of the strands deviates by no more than ±20% along their length and further includes embodiments in which the thickness/diameter of the strands deviates by no more than ±15% along their length.

The strands may be characterized by the shape of a lateral cross-section of a strand. Strands having various lateral cross-sectional shapes may be used, including circular and elliptical shapes. As discussed above, the printing methods make it possible to form uniform or substantially uniform strands, such that the lateral cross-sectional shape is maintained along the length of the strand.

The thickness and/or shape of the strands in a given layer can be, but need not be, the substantially the same as the thickness and/or shape of the strands in another layer. For example, the thickness of the strands in the scaffold may transition (e.g., continuously) from a first thickness value for stands in a layer at or near the bottom of the scaffold to a second (e.g., smaller) thickness value for strands in a layer at or near the top of the scaffold. Similarly, the lateral cross-sectional shape of the stands in the scaffold may transition from a first shape (e.g., circular) for strands in a layer at or near the bottom of the scaffold to a second shape (e.g., elliptical) for strands in a layer at or near the top of the scaffold. Other parameters of the scaffolds, e.g., strand spacing, may be substantially the same or different between different layers.

The 3D printing process is advantageous because it allows the user to control the porosity and pore interconnectivity of the scaffold. For example, for tissue growth scaffolds, the pore size, porosity and pore interconnectivity can be controlled to allow for the seeding of the cells and for efficient delivery of nutrients into and removal of waste from the interior of the scaffold. The optimal pore size, porosity and pore interconnectivity will depend on the cells being seeded and grown. Generally, it is desirably for the pores to have diameters of at least 2 times (e.g., 2-5 times) the diameter of the cells being seeded. For the purposes of this disclosure, pore diameter refers to a two-dimensional cross-sectional diameter, as viewed from above. Thus, the pore diameter for a given lateral cross-section of the scaffold could be determined by taking a lateral cross-section through the scaffold and imaging the surface of that section using, for example a high-resolution SEM. The average/median pore diameter for that layer could then be calculated from the image. An average/median pore diameter for the scaffold could be calculated by imaging a sufficient number of cross-sectional cuts through the scaffold. If the shape of a pore is not regular and symmetric, the diameter can be taken as the largest dimension across the pore. By way of illustration, in some embodiments of the present scaffolds, the average/median pore diameter is in the range from about 50 to about 1000 μm. This includes embodiments in which the average/median pore diameter is in the range from about 100 to about 500 μm. This further includes embodiments in which the average/median pore diameter is in the range from about 200 μm to about 1000 μm or from about 300 μm to about 400 μm. The shape and pore diameter of individual pores within the scaffold may be substantially the same (although they need not be).

The porosity of the scaffold refers to the percentage of void space within the scaffold. Some embodiments of the present scaffolds have a porosity of at least 50% (e.g., from about 50% to about 60%). This includes embodiments in which the porosity is at least about 60%, at least about 70%, at least about 80% or at least about 90% (e.g., from about 60% to about 95%). For the purposes of the present disclosure, the porosity values are measured via mercury intrusion porosimetry using a mercury porosimeter, following the method described in reference (13).

After the scaffold structure has been printed, it can undergo various post-extrusion treatments. For example, the soy proteins can be dehydrated by exposing the scaffold to an alcohol, such as ethanol. Other post-extrusion treatments include removing water from the scaffold via a dehydrothermal treatment; freeze-drying the scaffold such that water is removed via the sublimation of water frozen on the strand surfaces; and/or chemically or enzymatically crosslinking the soy protein chains in the denatured soy proteins. In some embodiments, the soy proteins are crosslinked with a relative crosslinking density of at least 0.1. This includes scaffolds in which the soy proteins have a relative crosslinking density of at least 0.2 or 0.3. The soy protein chains of the denatured soy proteins can be crosslinked by molecules capable of reacting with functional groups on the soy protein chains, such as —NH₂, —OH and —SH groups to form covalent linkages between the chains. In some embodiments, the crosslinkers are enzymatic crosslinkers. In some embodiments, the crosslinkers are non-enzymatic chemical molecules. Transglutaminase is one example of an enzymatic crosslinker. Organic molecules, such as carbodiimides (e.g., 1-ethyl-3-(3-dimethylaminopropyl)-carbodiimide) and N-hydroxysuccinimide are examples of non-enzymatic, chemical crosslinkers.

In some embodiments of the scaffolds, the strands in each layer are printed in a regular pattern, wherein the strands are spaced apart and aligned along their longitudinal axes. By rotating the scaffold structure between the extrusion of adjacent layers in such structures, the angle, θ, defined by the longitudinal axes of the strands in adjacent layers can be controlled such that it is in the range of 0°<θ≦90°. This allows for the formation of longitudinal pores in the form of a channel having a desired median pore diameter in the scaffold. The formation of such regular strands and channels within a printed scaffold is illustrated schematically in FIG. 1A, top panel. As shown in this figure, the strands can be characterized by an x-axis strand thickness (t) and a z-axis strand thickness (z). The layers can be characterized by a layer spacing (s) and a strand spacing (w). The relative orientation of two layers can be characterized by the angle θ. The leftmost image in this panel illustrates two layers having a θ of 45°. The middle image in the panel illustrates two layers having a θ of 90°. The rightmost image in the panel shows a cross-sectional view of the middle image. As shown in FIG. 1A, the angle θ is defined as the angle between the longitudinal axes of the strands in one layer and the longitudinal axes of the strands in an adjacent layer. This angle can range from 0° to 90°. However, provided that the angle is >0°, that is, provided the strands in adjacent layers are not aligned along their longitudinal axes, three-dimensional pores will be defined by neighboring strands in adjacent layers of the vertical stack. In some embodiments of the scaffolds, θ is in the range of 0°<θ≦90°. This includes embodiments in which θ is in the range of 20°≦θ≦90°, further includes embodiments in which θ is in the range of 45°≦θ≦90° and still further includes embodiments in which θ is in the range of 75°≦θ≦90°.

Typical x-axis and z-axis strand thicknesses for the present scaffolds include, but are not limited to, those in the range from 100 μm to 1000 μm (e.g., 100 μm to 600 μm; 150 to 300 μm). Typical strand spacings (w) in a scaffold with regularly patterned strands include those in the range from about 50 to about 1000 μm (e.g., from about 100 to about 500 μm, including from about 200 to about 300 μm).

A porous material intended for use as a tissue growth scaffold is also desirably sufficiently robust to support the viability of cells cultured thereon and, as such, is desirably elastic or viscoelastic. Robustness can be determined by the compressive modulus of the material. The compressive modulus of the scaffolds recited herein are measured as describe in the examples, below. The scaffolds can be made mechanically robust by, for example, increasing the degree of cross-linking between the soy proteins and/or increasing θ. In some embodiments, the scaffolds have a compressive modulus of at least 100 Pa. This includes embodiments having a compressive modulus of at least 1000 Pa, at least 2000 Pa and at least 4000 Pa. For example, the compressive moduli for the scaffolds can be in the range from about 1000 to about 5000 Pa. This includes embodiments in which the compressive moduli is in the range from about 100 Pa to about 1000 Pa or from about 100 Pa to about 200 Pa. The compressive moduli can refer to scaffolds in which the soy protein chains of the denatured soy proteins have not been crosslinked by enzymatic crosslinkers or non-enzymatic crosslinkers. In some embodiments, the compressive moduli refers to scaffolds in which the soy protein chains of the denatured soy proteins have not been crosslinked by a non-enzymatic crosslinker such as 1-ethyl-3-(3-dimethylaminopropyl)-carbodiimide)/N-hydroxysuccinimide and as such, the soy protein chains will not be crosslinked via the amide linkages provided by these crosslinkers.

The Example below illustrates the methods of making and using the scaffolds.

### Example

This Example focuses on the Bioplotting of a denatured soy protein, chosen due to the thermoplastic nature of the protein components (14), as well as the initial biocompatibility demonstrated both in vitro with different cell types (15-17) and in vivo (18).

Soy protein is a plant-based material with a wide range of structural and mechanical properties depending on the processing treatment (14). The degradation properties of soy biomaterials can be altered through different crosslinking treatments or fabrication techniques (16, 19, 20). Flexibility in material properties allows soy to be fabricated into a variety of structures including thin films (21), granules (17, 18, 22), hydrogels (23, 24), and scaffolds (16, 25). This Example illustrates the ability of the present methods to fabricate soy protein scaffolds in a reproducible manner with optimized printing parameters.

Methods and Materials

Mass Extrusion

Denatured soy protein isolate containing 87.6% protein as determined by combustion method (26) (Solae LLC, St. Louis, Mo., USA) was mixed with milliQ (MQ) water in varying wt. % to form a slurry. 1 wt. % antibiotic/antimycotic solution (Invitrogen, Carlsbad, Calif., USA) was added to all slurries. Glycerol (Sigma-Aldrich, St. Louis, Mo., USA) was added to 20 wt. % soy protein slurries as a plasticizer. Slurries were sieved sequentially through an autoclaved 297 μm pore size sieve (#50 mesh) and then an autoclaved 105 μm pore size sieve (#140 mesh) to remove non-dissolved particulates, forming a homogeneous paste. Sieved pastes were spread into a polyethylene (PE) syringe cartridge attached to a 200 μm PE tip and loaded into a 3D-Bioplotter (EnvisionTec GmbH, Germany) at room temperature. Slurries were extruded into a petri dish of known weight at a pressure of 3 bars for 2 seconds, and the ejected masses were weighed (N=3 measurements per temperature). Mass flow rate was determined by dividing the weight of the extruded slurry with the amount of time of extrusion (2 seconds). The temperature of the cartridge was then raised and held for 5 minutes prior to the next set of extrusions. Extrusions were performed for the temperature range of 22° C. to 70° C. Optimal mass flow rate for the soy protein slurry was determined by setting the pressure range between 0.5 bars to 3 bars at a fixed temperature of 27° C. To observe the effects of disulfide bond removal, 7.5 mM dithiothreitol (DTT, Sigma-Aldrich) was added to the 20 wt. % soy protein/4 wt. % glycerol solution. To visualize the macroscopic surface morphology of the printed strands, 2 wt. % trypan blue solution (Invitrogen) was added.

Scaffold Fabrication and Post-Treatments

Soy protein slurry composed of 20 wt. % soy protein/4 wt. % glycerol was prepared using the method described above. Individual boxes of 10 mm (length)×10 mm (width)×5 mm (height, 20 layers total) with 0=45° and 90° layer rotations (FIGS. 1A-1B) were fabricated onto a Teflon foil. Layer spacing (s) was set at 250 μm. Distance between strands was set at 1 mm to yield w=800 μm. Pressures applied to achieve the optimal flow rate through a 200 μm tip (to achieve t=200 μm) ranged between 0.8 bar to 2 bar. Plotting temperature and speed were 27° C. and 35 mm/sec, with a pre-flow delay of 0.1 seconds. All scaffolds were immersed in 95% ethanol for at least 3 hours prior to any further treatments. Following dehydration in ethanol, scaffolds were punched using a 6 mm biopsy punch to avoid edge effects. The four following treatment groups were considered: non-treated (NT), freeze-dried and dehydrothermal treated (FD-DHT), dehydrothermal treated only (DHT), and chemical crosslinked with carbodiimide crosslinking (EDC). Scaffolds that underwent no further treatment beyond 95% ethanol dehydration (NT group) were rinsed 3× and immersed in phosphate buffer solution (PBS) with calcium and magnesium (Hyclone, Logan, Utah, USA). A subset of scaffolds (FD-DHT group) were rinsed 3× with PBS, dried on 11 μm filter paper, frozen at −80° C. and dried to sublime water frozen on the strand surfaces. After lyophilization, scaffolds underwent dehydrothermal treatment (DHT) in a vacuum oven (VWR, Radnor, Pa., USA) at 105° C. for 24 hours under a vacuum of <100 mmHg. Scaffolds only treated with DHT (DHT group) were rinsed 3× in PBS and dried on 11 μm filter paper immediately before DHT. Scaffolds for chemical crosslinking (EDC group) were transferred to a solution of 375 mM 1-ethyl-3-(3 dimethylaminopropyl)carbodiimide (EDC) coupled with N-hydroxysuccinimide (NETS) at an EDC:NHS ratio of 5:2 (assuming 2 mmol carboxylic acid groups), all dissolved in 95% ethanol (27). All scaffold groups were immersed in PBS for at least 24 hours prior to experiments.

Surface Morphology Characterization

A Quanta 600F sFEG scanning electron microscope (SEM, FEI, Hillsboro, Oreg., USA) was used to visualize the scaffold structure and surface morphology of 90° scaffolds (N>2 per group). Cross-sections were obtained by cutting scaffolds using a scalpel. Hydrated scaffolds (NT and EDC groups and individual soy strands with and without addition of DTT) were immersed in 100% ethanol for at least 20 minutes and critical point dried. All scaffolds were coated with 9 nm of osmium prior to imaging. Dimensions of the various scaffolds including strand thickness t and pore width w for top and bottom layers as well as strand thickness z were measured using ImageJ with at least five measurements per sample and N=3.

Determination of Crosslink Density and Mass Loss

Relative crosslink density (XD), the inverse of swelling ratio directly related to the volume fraction of dry soy, was determined for the 45° scaffolds of the different groups (N=5-6) using a previously described method (28). Hydrated scaffolds were immersed in 90° C. water for 2 minutes. The scaffold was then pressed under a 1 kg weight between 11 μm filter papers for 20 seconds to remove water within the pores and weighed to obtain the wet mass (M_{w}). The wet scaffolds were dried overnight at 110° C. Density of crosslinks was then calculated using the wet and dry masses (M_{w} and M_{d} respectively) of the soy scaffold, the density of soy (ρₛ), and the density of water (ρ_{w}=1.00 g/cm³) using the following equation:

$$XD = \frac{\frac{{M}_{d}}{{ρ}_{s}}}{\frac{{M}_{d}}{{ρ}_{s}} + \frac{{M}_{w} - {M}_{d}}{{ρ}_{w}}}$$

The density of soy was determined for FD-DHT scaffolds using an AccuPyc 1340 helium pycnometer (Micromeritics, Norcross, Ga., USA) with N=3 taking the average of 6-8 independent measurements per sample. Average density calculated from FD-DHT scaffold results were used to reduce the likelihood of closed cell pores.

Mass loss of the scaffolds upon rinsing was determined by calculating the percent change between dry weights before and after rinsing. Hydrated NT and EDC scaffolds were freeze-dried before measuring starting dry weight (M_{b}). All scaffolds were individually rinsed 3× for 5 minutes in either PBS or MQ water, freeze-dried, then weighed to obtain mass after rinsing (Mₐ). Total percent mass loss was calculated as (M_{b}-Mₐ)/M_{b}*100%.

Mechanical Properties

Compression testing on 45° and 90° scaffolds was performed using a mechanical tester (JLW Instruments, Chicago, Ill., USA) using a previously described method (16). Briefly, scaffolds (N=5 per group) were compressed at 0.2 mm/min up to 45% strain, and the compressive modulus was determined by calculating the best fit slope of the linear (elastic) regime starting from the lowest strain outside the toe region (0.8% strain) to R²=0.95 (R²=0.99 for EDC scaffolds).

Cell Seeding

Scaffolds with 45° geometry were used to characterize cell attachment and growth. All scaffold groups were hydrated in PBS for at least 1 day prior to cell seeding. Scaffolds were sterilized in 70% ethanol and exposed to UV simultaneously for 30 minutes. After sterilization, scaffolds were dried on 11 μm filter paper before and after rinsing 3× in PBS with 5 minutes per rinse and incubated overnight in low glucose phenol red free DMEM media supplemented with 25 mM HEPES buffer, 2 mM L-glutamine, 10% FBS, and 1% antibiotic/antimycotic solution (all from Invitrogen, Carlsbad, Calif., USA).

Human mesenchymal stem cells (hMSC) from Lonza were passaged up to Passage 5 in MSC basal media with mesenchymal stem cell growth supplement, L-glutamine, and penicillin/streptomycin (Lonza, Walkersville, Md., USA). All cells were cultured at 37° C. in a 5% CO₂ humidified environment. Cells were trypsinized using 0.05% trypsin-EDTA (Invitrogen, Carlsbad, Calif., USA) and seeded onto the top layer of the printed scaffold at varying densities including 1×10⁴, 5×10⁴, and 1×10⁵ cells per scaffold. Cells were allowed to adhere to the scaffold for 90 minutes at 37° C. with 5% CO₂ before the addition of the phenol red free DMEM media described above. Scaffolds were cultured for 1 and 7 days.

Cell Seeding Efficiency, Proliferation, and Morphology on Various Scaffolds

Harvested scaffolds were cut into 4 pieces using microscissors and sonicated for 20 minutes in 1 mL of 0.02% (v/v) Triton X-100 (Bio-Rad, Hercules, Calif., USA) in MQ water. Samples were centrifuged at 15,000 rpm for 10 minutes at 4° C. A PicoGreen assay kit (Invitrogen, Carlsbad, Calif., USA) was used to quantify the amount of DNA in the supernatant. Aliquots of 100,000 cells taken during seeding were used to determine the amount of DNA per cell to convert DNA quantity to cell count. Cell seeding efficiency was calculated for the scaffolds seeded with 1×10⁵ cells. The seeding efficiency was defined as the percent difference between the numbers of cells present in the culture after 1 day (C) to the assumed amount of total cells seeded. The formula used was Seeding Efficiency (%)=C/(1×10⁵)*100%. Cell seeded scaffolds were fixed for one hour using 2% glutaraldehyde (Sigma-Aldrich) and 3% sucrose (J. T. Baker, Avantor, Center Valley, Pa.), dehydrated through graded ethanol, and critical point dried prior to imaging using SEM.

Statistical Analysis

All quantitative data from scaffold characterization analyses were reported as the mean with the positive error bar representing one standard deviation. Cell data was reported as the mean with the positive error bar representing standard error mean (SEM=standard deviation/n^{1/2}). Independent t-tests assuming unequal variance was used. One-way analysis of variance (ANOVA) with a Scheffe posthoc analysis (α=0.05) was performed with the post-treatment group as the fixed factor. P values <0.05 were considered statistically significant.

Results

Mass Extrusion of Soy Protein Slurry

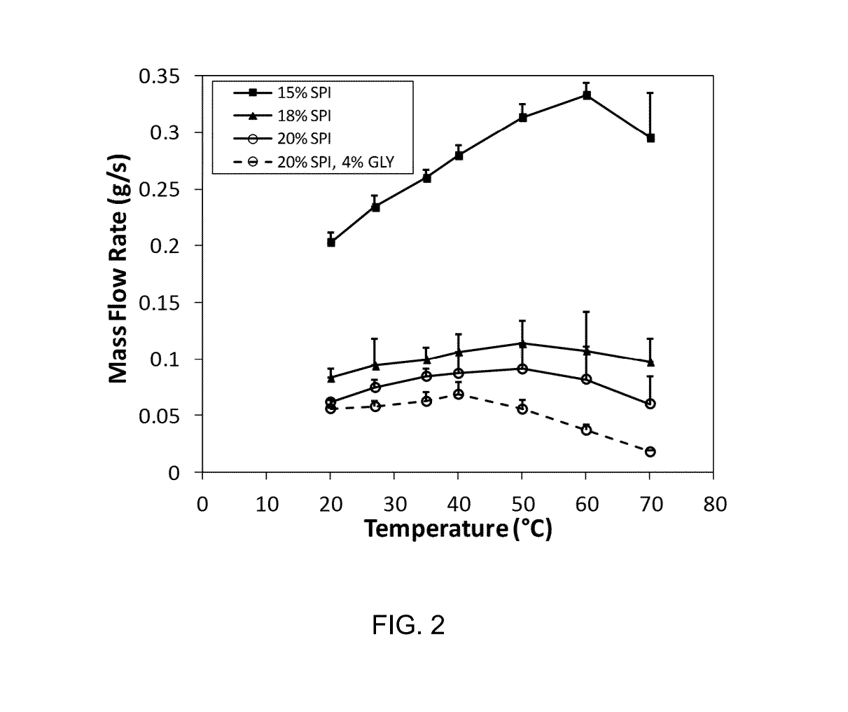

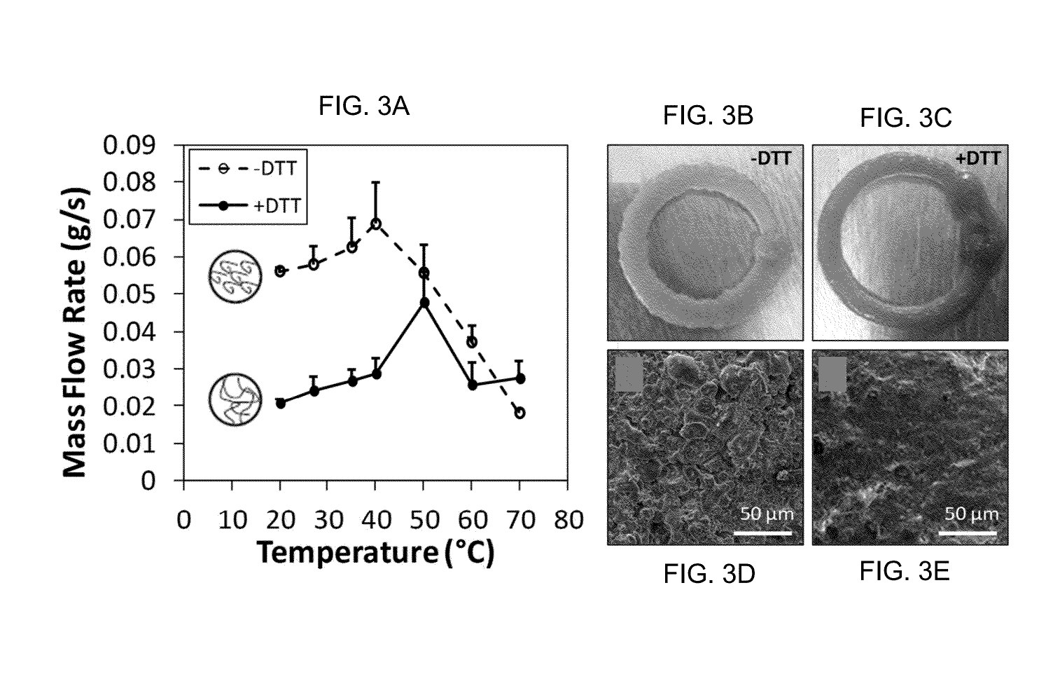

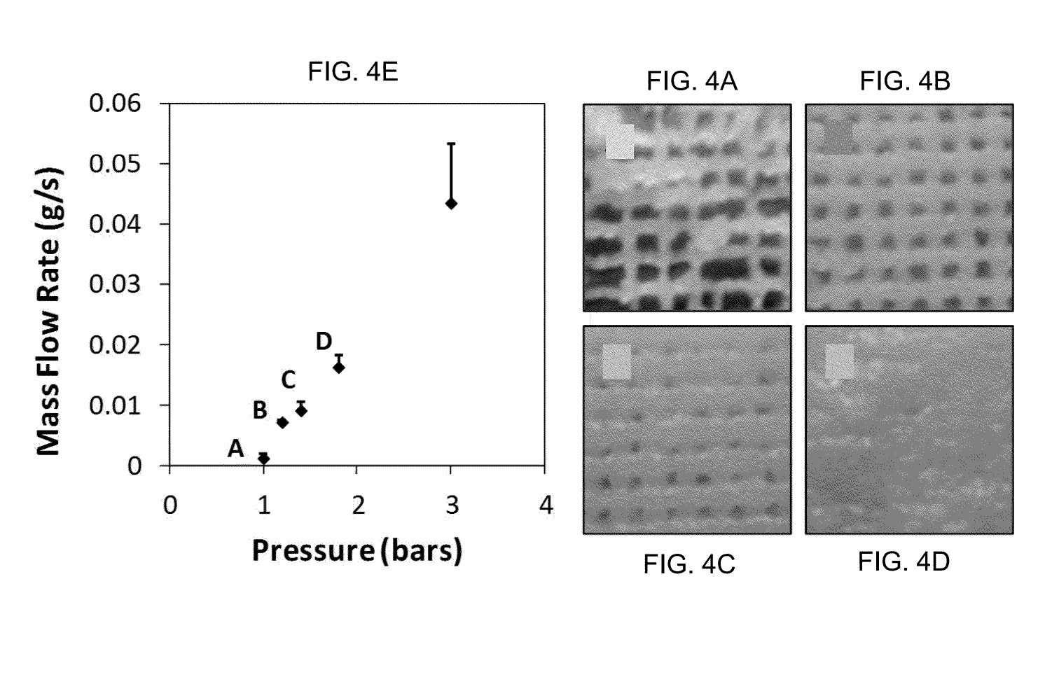

To determine an optimal soy slurry composition that could be used to fabricate 3D soy protein scaffolds with controlled pore architecture, the mass flow rate was measured for different slurry compositions up to the saturation point of soy protein at around 20 wt. % (FIG. 2). Increasing the total weight percent of soy protein decreased the mass flow rate. The addition of glycerol decreased the mass flow rate even further for all temperatures regardless of the percent weight of glycerol added up to 20 wt. % (data not shown). The mass flow rate of 20 wt. % soy and 4 wt. % glycerol slurry with DTT was decreased at all temperatures compared to slurry without DTT except at 70° C. (FIG. 3A). Macroscopic views of the strands showed that the slurry without DTT had limited uptake of trypan blue dye and rougher edges compared to the slurry with DTT (FIG. 3B, 3C). SEM images of the printed surfaces confirmed that the strand surface without DTT had rough surface topology compared to the smooth surface of the strand with DTT (FIG. 3D, 3E). The mass flow rate increased as extrusion pressure was increased (for the 20 wt. % soy and 4 wt. % glycerol slurry, FIG. 4E). The mass flow rate required to plot robust strands with z-pores and well-defined pore structures for this slurry composition was 0.0072±0.0002 g/s (FIG. 4B). Soy protein could not be deposited onto Teflon at pressures lower than optimal pressure (FIG. 4A). Increased pressures resulted in expansion of the strands and inability to form defined structures (FIG. 4C, 4D).

Scaffold Characterization

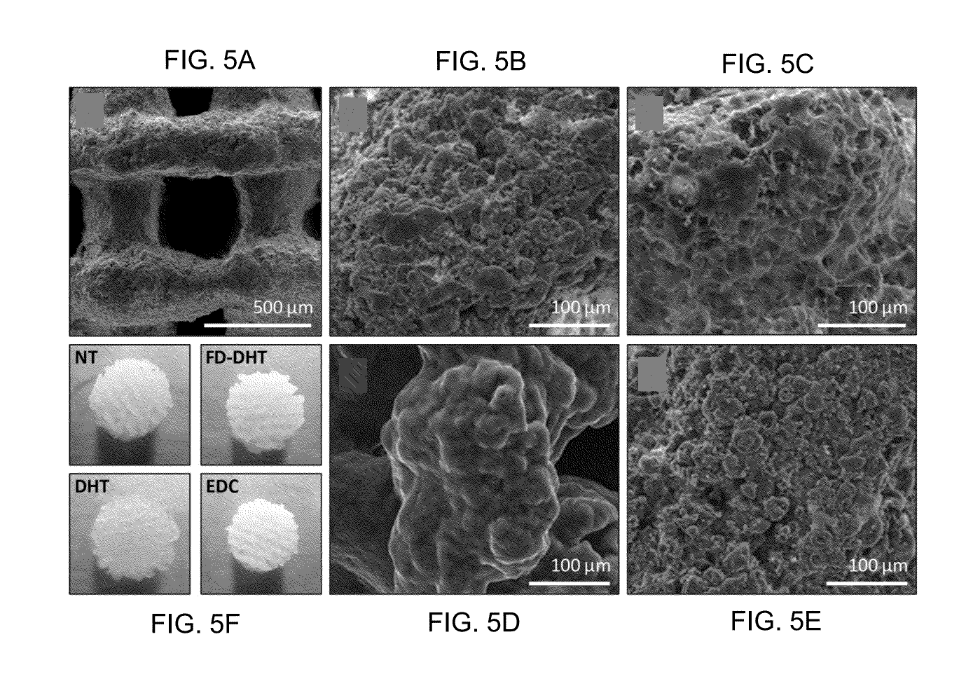

Fabricated scaffolds had well-defined pore geometries and varied surface morphologies depending on post-treatment type. The coloration of the scaffolds as viewed in the macroscopic images was darker yellow for the FD-DHT and DHT groups (FIG. 5F). Average diameters of the 45° soy scaffolds upon ethanol dehydration and immersing in PBS after 24 hours for the NT, FD-DHT, DHT, and EDC groups were 6.57±0.19 mm, 6.57±0.14 mm, 4.97±0.33 mm, and 6.09±0.04 mm respectively. SEM images of the scaffold struts showed differences in surface morphology after post-treatments. NT and EDC scaffolds had rough, dimpled surfaces with visible globules of various sizes on the order of 10 to 100 μm (FIG. 5B, 5E). The globules were connected homogeneously without any pores within the strand. Scaffolds that underwent dehydrothermal treatment had smooth surfaces with less ridges and interconnected globules (FIG. 5C, 5D). Rounded globules were not observed. The FD-DHT scaffold strand had pores within the surface connected by protein webs (FIG. 5C). A representative cross-section of the scaffolds (FIG. 5E) showed distinct stacking of strands in the z-axis with minor collapse of pores.

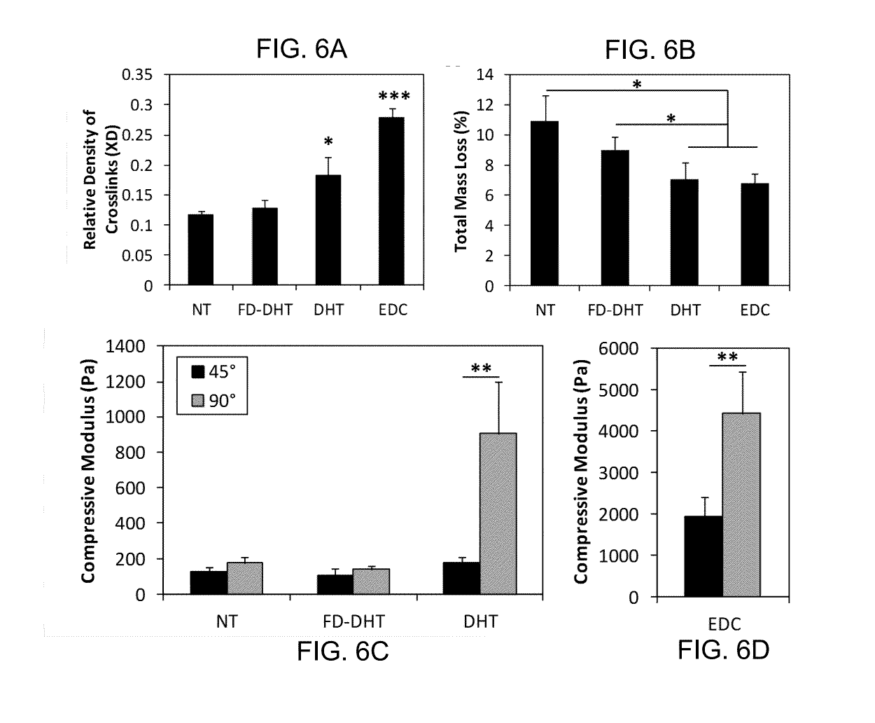

Post-treatment of the scaffolds affected the structural and mechanical properties and hence, the robustness of the scaffold (FIGS. 6A-6D). Measurements of strand thickness t from the top and bottom layers of a 90° scaffold showed that the strands expanded upon printing, decreasing the pore diameter w (FIG. 6A, 6B). The z-axis strand thickness was thinner than the strut thickness at the bottom of the NT, FD-DHT, and DHT scaffolds, implying that the strand shape became elliptical with the deposition of layers. DHT scaffolds had a significantly smaller strut thickness at the top of the scaffold compared to FD-DHT and EDC scaffolds (P<0.001). FD-DHT and DHT scaffolds expanded significantly with significantly smaller strut thicknesses at the top of the scaffold compared to the bottom (P<0.001). The pore width in the top layer for FD-DHT scaffolds was significantly larger compared to other groups (P<0.001). At the bottom of the scaffold, DHT scaffolds had significantly smaller pore width (P<0.001) compared to all other groups. The density of soy measured from helium pycnometry was 1.811±0.029 g/cm³. The relative density of crosslinks significantly increased with dehydrothermal treatment (P<0.05) and significantly increased by at least two-fold (P≦0.001) for the EDC group (FIG. 6A). ANOVA demonstrated that relative crosslink density across groups were significantly different (P<0.001). The mass loss of scaffolds rinsed in PBS ranged between 6-12%, with significant difference (P=0.001) between all groups as determined by ANOVA (FIG. 6B). Both DHT and EDC groups were significantly different from NT and FD-DHT groups (P<0.05). Similar total mass loss percent was observed for scaffolds rinsed in water, with the exception of the NT group which degraded entirely during the rinses. However, once rinsed in PBS, scaffold mass remained constant up to 30 days when incubated in PBS at 37° C. for all groups (data not shown). The compressive modulus was on the same order of magnitude for NT and FD-DHT groups for both 45° and 90° scaffolds and 45° DHT scaffolds (FIG. 6C). 90° DHT scaffolds had approximately four-fold higher compressive modulus. The compressive modulus was on the order of kPa for the EDC scaffolds, and 90° scaffolds had double the modulus compared to the 45° scaffolds (FIG. 6D). The compressive modulus was significantly different between 45° and 90° scaffolds for the DHT and EDC groups (P<0.01). From the results, scaffold groups could be identified in the following order from least to most robust: NT, FD-DHT, DHT, and EDC. Scheffe post-hoc analysis identified the EDC scaffolds to have significantly different crosslink density and mechanical properties compared to all other groups.

Cell Seeding, Proliferation, and Morphology in Printed Scaffolds

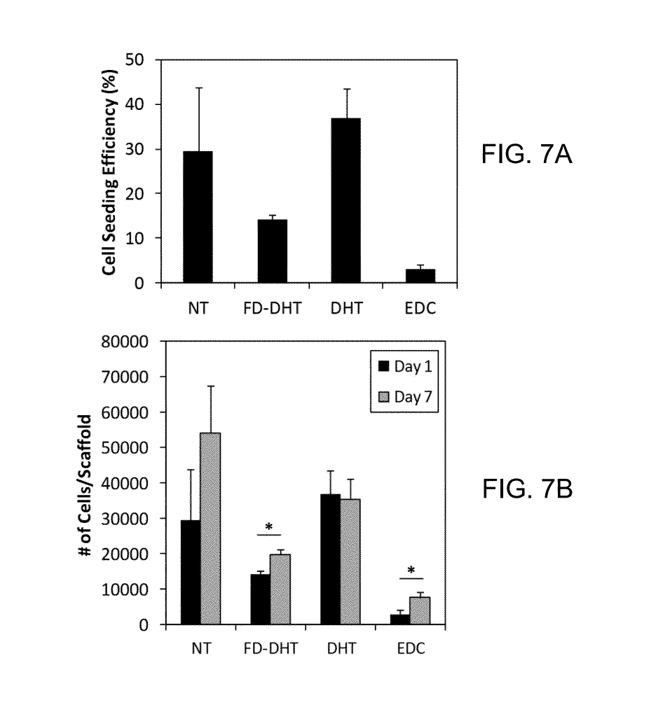

Cell seeding efficiencies and proliferation rates were varied between the post-treatment groups (FIGS. 7A-7B). The cell seeding efficiency was significantly different across all groups (P<0.01) as determined by ANOVA. Average seeding efficiencies for NT, DHT, FD-DHT, and EDC groups were 29.4±14.4%, 37.0±6.4%, 14.1±1.2%, and 3.0±1.1%, respectively (FIG. 7A). The cell seeding efficiency for FD-DHT, DHT, and EDC groups were significantly different from each other (P<0.05 for all combinations). The deviation in seeding efficiency for the NT group rendered it not statistically different from the other groups. The cell seeding efficiencies for all the groups did not change with varying cell seeding densities (data not shown). Cell viability was maintained across all groups, and proliferation was observed for cells seeded on the NT, EDC and FD-DHT groups. The average number of cells increased two-fold for cells seeded on both NT and EDC scaffolds. At day 1, the cells attached to the NT, FD-DHT, and DHT scaffolds appeared spread and elongated with fibroblast-like morphology on the strands and at the strand junctions; however, the cells did not bridge the pores for any of the scaffold groups. Cells attached to the EDC scaffolds appear rounded. Cell distribution across the scaffolds was similar for both days 1 and 7 (data not shown).

Discussion

This work demonstrated for the first time the ability to fabricate 3D soy protein porous structures via Bioplotting. A major challenge for Bioplotting natural biopolymers is the difficulty in reproducibly printing struts that hold shape and maintain scaffold structure. Pressures needed to print slurries are variable due to moisture content differences from batch to batch. This study used the mass flow rate (measured using the Bioplotter) as a reliable parameter for determining the ability to produce scaffolds with consistent self-supporting architectures, independent of soy slurry conditions. A mass flow rate of 0.0072±0.0002 g/s of soy protein produced well-defined scaffold constructs. To determine how this parameter varied with different slurry compositions, the mass flow rate was measured for different soy protein/glycerol concentrations. For all soy protein concentrations, mass flow rate increased and then decreased at temperatures between 40-60° C. (FIG. 2). This trend may be due to increasing mobility of protein chains as the temperature increases up to a solidification point (29), after which the flow rate starts to decrease due to a greater resistance to flow as the slurry continues to solidify (30, 31). The lowest flow rate for all temperatures was achieved with a slurry comprised of 20% soy protein and 4% glycerol slurry (FIG. 2).

To understand the molecular forces involved during printing for this system, DTT, a disulfide bond reducing agent, was added to 20% soy/4% glycerol slurries. The addition of DTT caused a lower mass flow rate, and the texture of the printed strands were smoothened (FIGS. 3A, 3C). This demonstrates that disulfide bonding is a significant molecular force involved in maintaining structural integrity in soy protein gels (32) and is needed to create stable and robust 3D plotted soy structures. The peak observed at 50° C. in slurries with DTT may have been caused by relaxation of chains held together by hydrogen bonding and hydrophobic interactions which are not affected by DTT (32). The lower mass flow rates of slurries containing DTT is expected based on reptation theory (33); the proteins solubilize and unravel without disulfide linkages (32), which allows for increased packing and entanglement of chains in a confined space (within the Bioplotting needle) causing a greater resistance to flow (FIG. 3A). Although there could be this increased entanglement of protein chains, the DTT-containing slurries could not solidify since the gelation mechanism of soy protein requires both physical and chemical crosslinks (dominated by disulfide bonding) (30).

Printing parameters including printing speed, layer spacing, and printing temperature were optimized for soy protein slurries to produce well-defined porous constructs. As mentioned previously, the relationship between pressure and mass flow rate may shift along the pressure axis due to varying slurry moisture content between batches. For instance, slurries with slightly less moisture content would require greater pressure to achieve a flow rate of 0.0072 g/s. However, regardless of the pressure needed for extrusion, the mass flow rate necessary to produce optimal scaffolds remains constant in a narrow range. Soy protein was unable to be deposited onto the Teflon foil at pressures lower than optimal, and at higher pressures (even just 0.2 bars away from the optimal pressure) the strands overflowed into the pores (FIGS. 4C, 4D). These studies demonstrate that the printing speed and extrusion pressure are variables to adjust when plotting natural biopolymers. The proper method to optimize these parameters first requires the determination of a printing speed that allows for sufficient drying into a discernible layer without clogging the printing nozzle, while also limiting expansion of the printed strands. With a set speed, the pressure can then be adjusted for optimal strand formation. The flow rate at this optimal speed and pressure can be measured and maintained during all further scaffold printing processes to ensure reproducibility. This quality control method may also be applied to the plotting of other soft materials with similar viscoelastic and drying/solidifying properties into liquid medium or air.

To fabricate soy protein scaffolds with well-defined pores, the layer spacing during printing was set to be greater than the strand size (nozzle tip) since the material expanded laterally upon extrusion (FIG. 6A, 6B); layer spacing less than 250 μm would cause layers to congeal together. To avoid issues with uneven gelation, scaffolds were printed at 27° C. but could have been printed at room temperature or at 37° C. since mass flow rate remained constant (FIG. 2). The ability to successfully print at these temperatures may allow for direct incorporation of growth factors and drugs within the scaffold struts during the printing process, as opposed to the more common method of post-coating the polymer struts with growth factors/drugs after fabrication (34, 35). To increase stability of the strands upon printing, ethanol treatment was used to further dehydrate the proteins. Electrostatic interactions between soy protein and PBS and media ions promote protein aggregation (30, 32). Therefore, scaffold structure is well maintained in physiologically relevant conditions (30) with minimal degradation observed after initial washing for up to 30 days (data not shown). The electrostatic interactions are necessary for the scaffold to retain shape, since non-crosslinked scaffolds dissolved when immersed in water.

Thermal and chemical post-treatments were applied to further increase scaffold robustness. Differences in structural and mechanical properties across the various post-treatments demonstrated the ability to tune the material properties of the printed soy protein scaffolds. Surface texture and scaffold size were the two main structural properties that underwent morphological changes. DHT treatment alone caused smoothening of microscopic texture through further molecular modifications, namely formation of hydrogen and disulfide bonds under heat (32) (FIG. 5D). Shrinking of struts at the top of the scaffold also occurred with both FD-DHT and DHT groups (FIG. 6A), indicating that residual moisture retained before treatment could affect size. Uneven heating at the top and bottom of the scaffold could have also caused thicker strand thicknesses at the bottom. The trends in increasing crosslink density and mechanical properties with decreasing total mass loss corresponded across the non-treated, thermally treated, and chemically treated scaffolds as expected (FIGS. 6A-6D). Freeze-drying before DHT significantly decreased the crosslink density, mass loss, and mechanical properties of the scaffolds compared to the DHT group, most likely due to the presence of pores within the struts after freeze-drying. Carbodiimide crosslinking that results in nonspecific peptide bond formation, created the highest crosslinked and therefore most robust scaffolds (27). The mechanical properties of soy protein can be varied across 2 orders of magnitude up to approximately 4 kPa. The compressive modulus of Bioplotted soy scaffolds was greater than soy protein scaffolds produced only by freeze-drying (not Bioplotted), which has moduli of approximately 50 Pa (16). 90° scaffolds had greater compressive modulus compared to 45° scaffolds (FIGS. 6C, 6D), which coincides with previously reported studies (36). The trend of increased mechanical properties for 90° scaffolds is more prominent for the more robust DHT and EDC scaffold groups. In particular, the average compressive modulus for the 90° scaffolds in the DHT group was significantly higher than the average for the 45° scaffolds. This may be due to the ability for greater evaporation of residual moisture (resulting from increased heat flux through the open pores) in the 90° scaffolds during DHT treatment leading to greater bonding at the junctions, resulting in a significantly higher mechanical strength in this geometry. The structural geometry did not affect the relative density of crosslinks or the total mass loss (data not shown).

The 45° scaffolds were chosen as an initial geometry for exploring cell attachment on soy scaffolds since limiting the amount of through pores has been shown to increase seeding efficiency (37). The seeding efficiencies were in similar ranges as reported with other Bioplotted scaffolds (between 25-45% (37)). The seeding efficiency may be further improved by exploring geometries with greater overlap of strands. Freeze-drying with DHT decreased the seeding efficiency due to the increase of small pockets or voids not large enough for cells to infiltrate, which decreased the available surface area for cells to attach. Seeding efficiency was also lower for EDC scaffolds likely due to reduced availability of epitopes for cell binding after crosslinking, which is confirmed by inability for cells to spread when attached to the surface. However, rounded cell morphology did not prevent cells from proliferating in the EDC group. Significant proliferation was observed for FD-DHT and EDC groups, and cell viability was maintained across the NT and DHT groups. DHT modification of the soy protein scaffolds maintained cell viability but resulted in limited cell proliferation. This result implies that although seeding density was lower for the FD-DHT and EDC groups, the cell density was still sufficient to support cell growth. At both days 1 and 7, the cells for all scaffold groups were homogeneously scattered throughout the entire scaffold structure. Differences in adhesion and proliferation between the non-treated and DHT-treated scaffolds are therefore likely due to different cell-surface interactions, mechanical, and degradation properties and not due to the dispersion of cells within the scaffold.

The results from these studies demonstrate that reproducible 3D soy protein scaffolds can be fabricated via 3D Bioplotting. The slurry flow rate was determined to be a parameter that could be used to predict the printability of soft materials, as well as serve as a measurement for quality control purposes. The ability to print soy protein is particularly advantageous since the processing can be done at either room temperature or 37° C. without the use of organic solvents, allowing for the possibility of incorporating drugs or growth factors during the printing process.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more”. Still further, the use of “and” or “or” is intended to include “and/or” unless specifically indicated otherwise.

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

### REFERENCES

1. Hogue M E, Chuan Y L, Pashby I. Extrusion based rapid prototyping technique: An advanced platform for tissue engineering scaffold fabrication. Biopolymers. 2012; 97(2):83-93.
2. Pfister A, Landers R, Laib A, Hubner U, Schmelzeisen R, Mulhaupt R. Biofunctional Rapid Prototyping for Tissue-Engineering Applications: 3D Bioplotting versus 3D Printing. J Polym Sci A Polym Chem. 2003; 42(3):624-38.
3. Sachlos E, Czernuszka J T. Making tissue engineering scaffolds work. Review on the application of solid freeform fabrication technology to the production of tissue engineering scaffolds. Eur Cell Mater. 2003; 5:29-40.
4. Peltola S M, Grijpma D W, Melchels F P W, Kellomaki M. A review of rapid prototyping techniques for tissue engineering purposes. Ann Med. 2008; 40(4):268-80.
5. Malafaya P B, Silva G A, Reis R L. Natural—origin polymers as carriers and scaffolds for biomolecules and cell delivery in tissue engineering applications. Adv Drug Deliv Rev. 2007; 59(4-5):207-33.
6. Maher P S, Keatch R P, Donnelly K, Paxton J Z. Formed 3D Bio-Scaffolds via Rapid Prototyping Technology. IFMBE Proceedings. 2008; 22.
7. Landers R, Hübner U, Schmelzeisen R, Müllhaupt R. Rapid prototyping of scaffolds derived from thermoreversible hydrogels and tailored for applications in tissue engineering. Biomaterials. 2002; 23(23):4437-47.
8. Liu C Z, Xia Z D, Han Z W, Hulley P A, Triffitt J T, Czernuszka J T. Novel 3D collagen scaffolds fabricated by indirect printing technique for tissue engineering. J Biomed Mater Res B Appl Biomater. 2008; 85B(2):519-28.
9. Yeong W-Y, Chua C-K, Leong K-F, Chandrasekaran M, Lee M-W. Indirect fabrication of collagen scaffold based on inkjet printing technique. Rapid Prototyping J. 2006; 12(4):229-37.
10. Sachlos E, Reis N, Ainsley C, Derby B, Czernuszka J T. Novel collagen scaffolds with predefined internal morphology made by solid freeform fabrication. Biomaterials. 2003; 24(8):1487-97.
11. Lam C X F, Mo X M, Teoh S H, Hutmacher D W. Scaffold development using 3D printing with a starch-based polymer. Mater Sci Eng C Mater Biol Appl. 2002; 20(1-2):49-56.
12. Rücker M, Laschke M W, Junker D, Carvalho C, Schramm A, Müllhaupt R, et al. Angiogenic and inflammatory response to biodegradable scaffolds in dorsal skinfold chambers of mice. Biomaterials. 2006; 27(29):5027-38.
13. Pappacena K E, Gentry S P, Wilkes T E, Johnson M T, Xie S, Davis A, et al. Effect of pyrolyzation temperature on wood-derived carbon and silicon carbide. J Eur Ceram Soc. 2009; 29(14):3069-77.
14. Wool R P, Sun X S. Bio-based polymers and composites. Burlington: Elsevier Academic Press; 2005.
15. Silva G A, Vaz C M, Coutinho O P, Cunha A M, Reis R L. In vitro degradation and cytocompatibility evaluation of novel soy and sodium caseinate-based membrane biomaterials. J Mater Sci Mater Med. 2003; 14(12):1055-66.
16. Chien K B, Shah R N. Novel soy protein scaffolds for tissue regeneration: Material characterization and interaction with human mesenchymal stem cells. Acta Biomater. 2012; 8(2):694-703.
17. Santin M, Morris C, Standen G, Nicolais L, Ambrosio L. A new class of bioactive and biodegradable soybean-based bone fillers. Biomacromolecules. 2007; 8(9):2706-11.
18. Merolli A, Nicolais L, Ambrosio L, Santin M. A degradable soybean-based biomaterial used effectively as a bone filler in vivo in a rabbit. Biomed Mater. 2010; 5(1):15008. Epub 2010 Feb. 3.
19. Song F, Tang D-L, Wang X-L, Wang Y-Z. Biodegradable Soy Protein Isolate-Based Materials: A Review. Biomacromolecules. 2011; 12(10):3369-80.
20. Vaz C M, De Graaf L A, Reis R L, Cunha A M. In vitro degradation behaviour of biodegradable soy plastics: effects of crosslinking with glyoxal and thermal treatment. Polym Degrad Stab. 2003; 81(1):65-74.
21. Chen L, Remondetto G, Rouabhia M, Subirade M. Kinetics of the breakdown of cross-linked soy protein films for drug delivery. Biomaterials. 2008; 29(27):3750-6.
22. Giavaresi G, Fini M, Salvage J, Nicoli Aldini N, Giardino R, Ambrosio L, et al. Bone regeneration potential of a soybean-based filler: experimental study in a rabbit cancellous bone defects. J Mater Sci Mater Med. 2009; 21(2):615-26.
23. Song F, Zhang L-M. Gelation Modification of Soy Protein Isolate by a Naturally Occurring Cross-Linking Agent and Its Potential Biomedical Application. Ind Eng Chem Res. 2009; 48(15):7077-83.
24. Santin M, Ambrosio L. Soybean-based biomaterials: preparation, properties and tissue regeneration potential. Expert Rev Med Devices. 2008; 5(3):349-58.
25. Guan J, Porter D, Tian K, Shao Z, Chen X. Morphology and mechanical properties of soy protein scaffolds made by directional freezing. J Appl Polym Sci. 2010; 118(3):1658-65.
26. AOAC. Official Methods of Analysis of AOAC International. 17th ed.; AOAC International: Gaithersburg, Md., 2000.
27. Damink L H H O, Dijkstra P J, van Luyn M J A, van Wachem P B, Nieuwenhuis P, Feijen J. Cross-linking of dermal sheep collagen using a water-soluble carbodiimide. Biomaterials. 1996; 17(8):765-73.
28. Vickers S M, Squitieri L S, Spector M. Effects of Cross-linking Type II Collagen-GAG Scaffolds on Chondrogenesis In Vitro: Dynamic Pore Reduction Promotes Cartilage Formation. Tissue Eng. 2006; 12(5):1345-55.
29. Hiemenz P C, Lodge T P. Polymer Chemistry: Second Edition. Boca Raton: CRC Press; 2007. 381-418 p.
30. Hermansson A M. Soy Protein Gelation. J Am Oil Chem Soc. 1986; 63(5):658-66.
31. Utsumi S, Kinsella J E. Structure-Function Relationships in Food Proteins: Subunit Interactions in Heat-Induced Gelation of 7S, 11S, and Soy Isolate Proteins. J Agric Food Chem. 1985; 33(2):297-303.
32. Utsumi S, Kinsella J E. Forces Involved in Soy Protein Gelation: Effects of Various Reagents on the Formation, Hardness, and Solubility of Heat-Induced Gels Made from 7S, 11S, and Soy Isolate. J Food Sci. 1985; 50(5):1278-82.
33. Fetters L J, Lohse D J, Richter D, Witten T A, Zirkel A. Connection between Polymer Molecular Weight, Density, Chain Dimensions, and Melt Viscoelastic Properties. Macromol. 1994; 27(17):4639-47.
34. Laschke M W, Rücker M, Jensen G, Carvalho C, Müllhaupt R, Gellrich N C, et al. Incorporation of growth factor containing Matrigel promotes vascularization of porous PLGA scaffolds. J Biomed Mater Res A. 2008; 85A(2):397-407.
35. Lindhorst D, Tavassol F, von See C, Schumann P, Laschke M W, Harder Y, et al. Effects of VEGF loading on scaffold-confined vascularization. J Biomed Mater Res A. 2010; 95A(3):783-92.
36. Moroni L, de Wijn J R, van Blitterswijk C A. 3D fiber-deposited scaffolds for tissue engineering: Influence of pores geometry and architecture on dynamic mechanical properties. Biomaterials. 2006; 27(7):974-85.
37. Sobral J M, Caridade S G, Sousa R A, Mano J F, Reis R L. Three-dimensional plotted scaffolds with controlled pore size gradients: Effect of scaffold geometry on mechanical performance and cell seeding efficiency. Acta Biomater. 2011; 7(3):1009-18.

What is claimed is:

1. A method of forming a porous biopolymer-containing scaffold, the method comprising:
extruding a slurry comprising a biopolymer in the form of a first layer, the first layer comprising a plurality of strands; and
extruding the slurry in the form of one or more additional layers, each additional layer being vertically stacked upon the previously extruded layer and comprising a plurality of strands,
wherein the mass flow rate of the slurry is maintained at a substantially constant rate during extrusion by adjusting one or both of the extrusion pressure and extrusion speed during extrusion.

2. The method of claim 1, wherein the extrusion is accomplished via extrusion-based rapid prototyping.

3. The method of claim 1, wherein the biopolymer is denatured soy protein.

4. The method of claim 3, wherein the amount of the denatured soy protein in the slurry is in the range of from about 15 wt % to about 20 wt % and further wherein, the slurry further comprises a plasticizer.

5. The method of claim 4, wherein the amount of the plasticizer in the slurry is about 4 wt %.

6. The method of claim 3, wherein the mass flow rate is about 0.0072 g/s.

7. The method of claim 1, wherein the extrusion is carried out at a temperature in the range of from about room temperature to about 40° C.

8. The method of claim 1, wherein the slurry further comprises a growth factor or a drug.

9. The method of claim 1, further comprising solidifying the extruded porous biopolymer-containing scaffold via a post-extrusion treatment.

10. The method of claim 9, wherein the solidification is accomplished via a dehydrothermal treatment.

11. The method of claim 10, further comprising freeze-drying the extruded porous biopolymer-containing scaffold prior to the dehydrothermal treatment.

12. The method of claim 9, wherein the solidification is accomplished via chemically or enzymatically crosslinking the biopolymer in the extruded porous biopolymer-containing scaffold.

13. The method of claim 1, wherein the extrusion is accomplished via extrusion-based rapid prototyping, wherein the biopolymer is denatured soy protein, and further wherein the method further comprises solidifying the extruded porous biopolymer-containing scaffold via a post-extrusion treatment.

14. The method of claim 13, wherein the amount of the denatured soy protein in the slurry is in the range of from about 15 wt % to about 20 wt % and further wherein, the slurry further comprises a plasticizer.

15. The method of claim 14, wherein the amount of the plasticizer in the slurry is about 4 wt %.

16. The method of claim 13, wherein the mass flow rate is about 0.0072 g/s.

17. The method of claim 13, wherein the extrusion is carried out at a temperature in the range of from about room temperature to about 40° C.

18. The method of claim 13, wherein the slurry further comprises a growth factor or a drug.

19. The method of claim 13, wherein the solidification is accomplished via a dehydrothermal treatment, optionally further comprising freeze-drying the extruded porous biopolymer-containing scaffold prior to the dehydrothermal treatment.

20. The method of claim 13, wherein the solidification is accomplished via chemically or enzymatically crosslinking the biopolymer in the extruded porous biopolymer-containing scaffold.
